(19) United States
(12) Patent Application Publication
(10) Pub. No.: US 2019/0060216 A1
(43) Pub. Date: Feb. 28, 2019

(54) ANTI-POLLUTION COMPOSITIONS CONTAINING BACILLUS COAGULANS

(71) Applicants: Muhammed Majeed, Edison, NJ (US); Kalyanam Nagabhushanam, East Windsor, NJ (US); Lakshmi Mundkur, Bangalore, IN (US); Shaheen Majeed, Springville, UT (US)

(72) Inventors: Muhammed Majeed, Edison, NJ (US); Kalyanam Nagabhushanam, East Windsor, NJ (US); Lakshmi Mundkur, Bangalore, IN (US); Shaheen Majeed, Springville, UT (US)

(21) Appl. No.: 16/117,146

(22) Filed: Aug. 30, 2018

(30) Foreign Application Priority Data
Aug. 31, 2017 (IN) 201741030867

(51) Int. Cl. A61K 8/99; A61Q 19/08; A61Q 17/04; A61K 35/742; A61P 39/06

(52) U.S. Cl. CPC A61K 8/99; A61Q 19/08; A61Q 17/04; A61K 2800/524; A61P 39/06; A61K 2800/522; A61K 35/742

(57) ABSTRACT

Disclosed are the anti-pollution effects of probiotic bacteria Bacillus coagulans MTCC 5856. More specifically the invention discloses the use of probiotic bacteria Bacillus coagulans MTCC 5856 in protecting mammalian skin against the harmful effects of UV and different environmental pollutants. The use of Bacillus coagulans MTCC 5856, as an antioxidant, skin rejuvenating and cleansing agent is also disclosed.

### CROSS REFERENCE TO RELATED APPLICATION

This is a conventional US patent application claiming priority from Indian Provisional application no. 201741030867 filed on 31 Aug. 2017, the details of which are being incorporated herein by reference.

### FIELD OF INVENTION

The present invention relates to probiotic compositions. More specifically, the present invention pertains to compositions comprising probiotic microorganism Bacillus coagulans MTCC 5856 for use as an anti-pollutant and skin rejuvenation/cleansing agent.

### DESCRIPTION OF PRIOR ART

Skin, the largest organ in the mammalian body, plays the role of a barrier by conferring protection from mechanical impacts and pressure, variations in temperature, micro-organisms, radiation and chemicals. However, the skin is exposed to a variety of pollutants thereby causing premature skin ageing, pigmentation spots, or acne or lead to more serious dermatological issues such as atopic dermatitis, psoriasis, and even skin cancer (English, J. S., R. S. Dawe, and J. Ferguson, Environmental effects and skin disease. Br Med Bull, 2003. 68: p. 129-42).

A recent report from WHO indicate that almost everybody is affected and over 3 million people in the world die due to increase in the levels of pollution. The main sources of pollution include particulate matter, polycyclic aromatic hydrocarbons (PAHs), volatile organic compounds (VOCs), nitrogen and sulfur oxides, carbon monoxide, ozone, and heavy metals (Baudouin, C., et al., Environmental pollutants and skin cancer. Cell Biol Toxicol, 2002. 18(5): p. 341-8). The toxic gases (CO2, CO, SO2, NO, NO2), low molecular weight hydrocarbons, persistent organic pollutants (e.g., dioxins), heavy metals (e.g., lead, mercury) and particulate matter (PM) form the primary pollutants which are formed from the source. Secondary pollutants, which include ozone (O3), NO2, peroxy acetyl nitrate, hydrogen peroxide and aldehydes are formed in the atmosphere through chemical and photochemical reactions involving primary pollutants (Kampa, M. and E. Castanas, Human health effects of air pollution. Environ Pollut, 2008. 151(2): p. 362-7).

The effect of pollution on the skin is manifold. In the presence of pollutants, the composition of normal microbiome of the skin is altered which lead to colonization with pathogenic organisms (Jo, J. H., E. A. Kennedy, and H. H. Kong, Topographical and physiological differences of the skin mycobiome in health and disease. Virulence, 2017. 8(3): p. 324-333). Pollution enhances the production of reactive oxygen species (ROS), which depletes the content of antioxidants in the skin. This causes disturbance in the redox balance, causing stress to the cells. As the pollutants permeate through the skin layers, they can activate the Aryl hydrocarbon receptor (AhR), which mediates the toxic effects of pollutants. The inflammatory cascade is activated by these alterations, which results in increased production of pro inflammatory cytokines such as interleukin (IL)-1 or IL 8, resulting in skin lesions and deterioration of skin appearance (Mancebo, S. E. and S. Q. Wang, Recognizing the impact of ambient air pollution on skin health. J Eur Acad Dermatol Venereol, 2015. 29(12): p. 2326-32).

Environmental pollutants like UVA up regulates the formation of matrix metalloproteinase (MMPs), enzymes that degrade the matrix protein's elastin and collagen, which, if not prevented, can result in marked reduction in skin elasticity and increased wrinkling (Risom, L., P. Moller, and S. Loft, Oxidative stress-induced DNA damage by particulate air pollution. Mutat Res, 2005. 592(1-2): p. 119-37; Moller, P. and S. Loft, Oxidative damage to DNA and lipids as biomarkers of exposure to air pollution. Environ Health Perspect, 2010. 118(8): p. 1126-36). UVA can penetrate deeper into the skin in comparison to UVB and contributes to photoaging, photocarcinogenesis and photodermatosis and increase oxidative stress in fibroblasts and cells which are deeper inside the skin. Blue light (light from mobile, TV, laptop/desktop screens) is reported to exert similar effect (Godley et al., Blue Light Induces Mitochondrial DNA Damage and Free Radical Production in Epithelial Cells, The Journal Of Biological Chemistry, 2005, 280(22):21061-21066).

Oxidative stress is defined as the imbalance in the redox characteristics of cellular environments resulting from (1) aberrant biochemical processes leading to the production of reactive species, (2) exposure to damaging agents (i.e., environmental pollutants and radiations), or (3) limited capabilities of endogenous antioxidant systems. Reactive oxygen and nitrogen species (ROS/RNS) produced under oxidative stress are known to damage all cellular biomolecules (lipids, sugars, proteins, and polynucleotides).

The following prior art documents, describe in detail the role of ROS/RNS the pathogenesis of different diseases:
a. Bickers D. R., Athar M “Oxidative stress in the pathogenesis of skin disease. The Journal of Investigative Dermatology. 2006; 126(12):2565-2575.
b. Franco R., Sanchez-Olea R., Reyes-Reyes E. M., Panayiotidis M. I. Environmental toxicity, oxidative stress and apoptosis: menage a trois. Mutation Research. 2009; 674(1-2):3-22.
c. Hodjat M., Rezvanfar M. A., Abdollahi M. A systematic review on the role of environmental toxicants in stem cells aging. Food and Chemical Toxicology. 2015; 86:298-308.
a. Negre-Salvayre A., Auge N., Ayala V., et al. Pathological aspects of lipid peroxidation. Free Radical Research. 2010; 44(10):1125-1171.
b. Roberts R. A., Smith R. A., Safe S., Szabo C., Tjalkens R. B., Robertson F. M. Toxicological and patho-physiological roles of reactive oxygen and nitrogen species. Toxicology. 2010: 276(2):85-94.

Cellular defence systems to prevent uncontrolled ROS increase include nonenzymatic molecules (glutathione, vitaminutes A, C, and E, and several antioxidants present in foods) and enzymatic scavengers of ROS, with superoxide dismutase (SOD), catalase (CAT), and glutathione peroxidase (GPX) being the best-known mechanisms.

In addition to such cellular defence mechanisms, extraneous agents to modulate cellular redox equilibrium are also known in the art. Combinatorial probiotic schemes (example, combination of Lactobacillus acidophilus and Bifidobacterium animalis) are shown to be anti-oxidants in Stancu C S et al, “Probiotics determine hypolipidemic and antioxidant effects in hyperlipidemic hamsters”, Mol Nutr Food Res. 2014 March; 58(3):559-68. Probiotics are also reported to be improve skin health and exert anti-pollution effects:
a. Kober et al., The effect of probiotics on immune regulation, acne, and photoaging International Journal of Women's Dermatology, Volume 1, Issue 2, June 2015, Pages 85-89
b. Jeb Gleason, 2018, [in-cosmetics Global] Anti-pollution, Probiotics and UV Protection Drive Protective Skin Care, https://www.gcimagazine.com/business/rd/claims/Anti-pollution-Probiotics-and-UV-Protection-Drive-Protective-Skin-Care--482202421.html accessed on 10 Aug. 2018.
c. Roudsari et al., Health Effects of Probiotics on the Skin, Critical Reviews in Food Science and Nutrition, Volume 55, 2015—Issue 9

However, technical problems do exist in using probiotic microorganisms for therapeutic purposes due to the common understanding that probiotic biological activities are strain specific. The genus-species-strain specific differences in biological activities are to be evaluated to link probiotics to specific health effects and also to enable accurate surveillance and epidemiological studies as indicated in Joint FAO/WHO Working Group Report on Drafting Guidelines for the Evaluation of Probiotics in Food London, Ontario, Canada, April 30 and May 1, 2002—See Section 3.1. Thus a superior probiotic which can exert an excellent anti-pollution effect and rejuvenate the skin is still warranted. The present invention overcomes the aforesaid technical problem by disclosing probiotic microorganism Bacillus coagulans MTCC 5856 as an effective anti-pollution agent and an excellent antioxidant.

It is the principle objective of the present invention to disclose the anti-pollution effects of probiotic microorganism Bacillus coagulans MTCC 5856 and as a skin rejuvenating/cleansing agent.

It is another objective of invention to disclose the antioxidant property of Bacillus coagulans MTCC 5856.

The present invention fulfils the aforesaid objective and provides further related advantages.

### Deposit of Biological Material

The deposit of biological material Bacillus coagulans bearing accession number MTCC 5856, mentioned in the instant application has been made on 19 Sep. 2013 at Microbial Type Culture Collection & Gene Bank (MTCC), CSIR-Institute of Microbial Technology, Sector 39-A, Chandigarh—160036, India.

### SUMMARY OF INVENTION

The present invention discloses the anti-pollution effects of probiotic bacteria Bacillus coagulans MTCC 5856 on the skin of mammals. More specifically the invention discloses the use of probiotic bacteria Bacillus coagulans MTCC 5856 in protecting the skin against the harmful effects of UV and different environmental pollutants. The use of Bacillus coagulans MTCC 5856, as an antioxidant, skin rejuvenating and cleansing agent is also disclosed.

Other features and advantages of the present invention will become apparent from the following more detailed description, taken in conjunction with the accompanying images, which illustrate, by way of example, the principle of the invention.

### BRIEF DESCRIPTION OF THE FIGURES

FIG. 1a is the graphical representation showing the decrease in ROS production by probiotic bacteria Bacillus coagulans MTCC 5856 in mouse fibroblast cells, exposed to UV-A.

FIG. 1b is the graphical representation showing the percentage ROS scavenging by probiotic bacteria Bacillus coagulans MTCC 5856 in mouse fibroblast cells, exposed to UV-A.

FIG. 2a is the graphical representation showing the decrease in ROS production by probiotic bacteria Bacillus coagulans MTCC 5856 in human keratinocytes, exposed to UV-B.

FIG. 2b is the graphical representation showing the percentage ROS scavenging by probiotic bacteria Bacillus coagulans MTCC 5856 in human keratinocytes, exposed to UV-B.

FIG. 3a is the graphical representation showing the decrease in ROS production by probiotic bacteria Bacillus coagulans MTCC 5856 in mouse fibroblast cells, exposed to sodium lauryl sulfate.

FIG. 3b is the graphical representation showing the percentage ROS scavenging by probiotic bacteria Bacillus coagulans coagulans MTCC 5856 in mouse fibroblast cells, exposed to sodium lauryl sulfate.

FIG. 4a is the graphical representation showing the decrease in ROS production by probiotic bacteria Bacillus coagulans MTCC 5856 in human keratinocytes, exposed to mixture of heavy metals

FIG. 4b is the graphical representation showing the percentage ROS scavenging by probiotic bacteria Bacillus coagulans coagulans MTCC 5856 in human keratinocytes, exposed to mixture of heavy metals

FIG. 5a is the graphical representation showing the increase in cell survival by probiotic bacteria Bacillus coagulans MTCC 5856 in human keratinocytes, exposed to mixture of benzpyrene (PAH) and UV irradiation

FIG. 5b is the graphical representation showing the percentage protection against cell death in human keratinocytes, exposed to mixture of benzpyrene (PAH) and UV irradiation by probiotic bacteria Bacillus coagulans coagulans MTCC 5856

FIG. 6a is the graphical representation showing the increase in cellular glutathione levels by probiotic bacteria Bacillus coagulans MTCC 5856 in human keratinocytes, exposed to UV-A.

FIG. 6b is the graphical representation showing the increase in cellular glutathione levels by probiotic bacteria Bacillus coagulans MTCC 5856 in human keratinocytes, exposed to UV-B.

FIG. 7a is the graphical representation showing the increase in cellular superoxide dismutase activity by probiotic bacteria Bacillus coagulans MTCC 5856 in human keratinocytes, exposed to UV-A.

FIG. 7b is the graphical representation showing the increase in cellular superoxide dismutase activity by probiotic bacteria Bacillus coagulans MTCC 5856 in human keratinocytes, exposed to UV-B.

### DESCRIPTION OF THE MOST PREFERRED EMBODIMENTS

In the most preferred embodiments, the present invention discloses a composition containing probiotic bacteria Bacillus coagulans for protecting mammalian skin against the harmful effects of UV radiation and environmental pollutants. In a related embodiment, the probiotic bacteria Bacillus coagulans is present in the form of a spore or viable bacilli. In another related embodiment the environmental pollutants are selected from the list consisting of, but not limited to, particulate matter, polycyclic aromatic hydrocarbons (PAHs), volatile organic compounds (VOCs), detergents, nitrogen and sulfur oxides, carbon monoxide, ozone, and heavy metals. In another related embodiment, the probiotic bacteria confers skin protection by increasing the levels of anti-oxidants and decreasing ROS levels. In a related embodiment, the Bacillus coagulans strain is preferably Bacillus coagulans MTCC 5856. In yet another related embodiment the composition is formulated with pharmaceutically/cosmeceutically acceptable excipients, adjuvants, bases, diluents, carriers, conditioning agents, bioavailability enhancers, and preservatives and/or incorporated into formulations containing skin care ingredients and administered topically in the form of creams, gels, lotions, powder, serum, oil, suspensions, ointments, soaps, scrubs, emulsions, and compacts.

In another most preferred embodiment, the invention discloses a method for cleansing a rejuvenating mammalian skin exposed to environmental pollutants and UV radiation, said method comprising step of administering an effective dose of a composition containing probiotic bacterial Bacillus coagulans to mammals in need of such effect. In a related embodiment, the probiotic bacteria Bacillus coagulans is present in the form of a spore or viable bacilli. In another related embodiment the environmental pollutants are selected from the list consisting of, but not limited to, particulate matter, polycyclic aromatic hydrocarbons (PAHs), volatile organic compounds (VOCs), detergents, nitrogen and sulfur oxides, carbon monoxide, ozone, and heavy metals. In another related embodiment, the probiotic bacteria rejuvenates the skin by increasing the levels of anti-oxidants and decreasing ROS levels. In a related embodiment, the Bacillus coagulans strain is preferably Bacillus coagulans MTCC 5856. In yet another related embodiment the composition is formulated with pharmaceutically/cosmeceutically acceptable excipients, adjuvants, bases, diluents, carriers, conditioning agents, bioavailability enhancers, and preservatives and/or incorporated into formulations containing skin care ingredients and administered topically in the form of creams, gels, lotions, powder, serum, oil, suspensions, ointments, soaps, scrubs, emulsions, and compacts.

In yet another preferred embodiment, the invention discloses a composition containing probiotic bacteria Bacillus coagulans for use as an antioxidant. In a related embodiment, the composition containing probiotic bacteria Bacillus coagulans is used in the therapeutic management of mammalian cellular oxidative stress. In a related embodiment, the probiotic bacteria Bacillus coagulans is present in the form of a spore or viable bacilli. In a related embodiment, the Bacillus coagulans strain is preferably Bacillus coagulans MTCC 5856. In yet another related embodiment the composition is formulated with pharmaceutically/cosmeceutically acceptable excipients, adjuvants, bases, diluents, carriers, conditioning agents, bioavailability enhancers, and preservatives and/or incorporated into formulations containing skin care ingredients and administered orally or topically in the form of tablet, capsule, powder, emulsions, solution, creams, gels, lotions, powder, serum, oil, suspensions, ointments, soaps, scrubs, emulsions, and compacts, suited for nutraceutical, cosmeceutical and nutri-cosmetic applications.

Specific illustrative examples enunciating the most preferred embodiments are included herein below.

In vitro anti-pollution tests are based on cell models that are set up to reflect the in vivo state under laboratory conditions. They are usually carried out using keratinocyte or fibroblast cell lines. The impact of pollution on skin cells and the effect of the anti-pollution treatment are assessed by the quantification of specific markers and cell parameters.
1. Intracellular ROS accumulation is measured using a prefluorescent probe, which is oxidized by ROS and gives a fluorescent compound (Rosenkranz, A. R., et al., A microplate assay for the detection of oxidative products using 2′,7′-dichlorofluorescin-diacetate. J Immunol Methods, 1992. 156(1): p. 39-45)
2. The effect of anti-pollution treatment is reflected in the antioxidant capacity of the skin and the ability of antioxidants to neutralize harmful substances.
3. The effect of anti pollution treatment is also reflected as increase in cellular anti oxidant enzymes and decrease in inflammation

### Example 1

### Antipollution Effects of Bacillus coagulans MTCC 5856

### Methods

ROS assay: A cell permeable, non-fluorescent dye, 2′,7′-dichlorofluorescein diacetate (DCFH-DA) enters the cell and the acetate group on DCFH-DA is cleaved by cellular esterases, trapping the non-fluorescent DCFH inside the cell. Subsequent oxidation by reactive oxygen species generated by ferrous sulphate in the cells, yields the fluorescent DCF which can be detected at 485/520 Ex:Em wavelength. The scavenging activity of sample is indicated by the decrease in fluorescence when compared to the control without antioxidant.

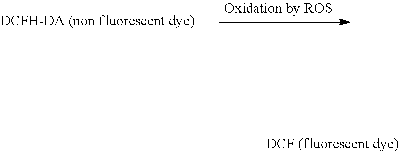

Human HaCaT keratinocyte cells/mouse fibroblast cells were maintained in DMEM containing 25 mM glucose with 10% heat-inactivated fetal calf serum with antibiotics at 37° C. and 5% CO₂. When the cells were 70-80% confluent, they were trypsinized, washed and seeded in 96 well plates at a density seeded at a density of 1×10⁴ cells/well. Cells were allowed to adhere and form a monolayer for 24 hours. Cells were pretreated with varying non toxic concentrations of Bacillus coagulans MTCC 5856 in PBS for 60 minutes before exposing to the pollutant. Cells were exposed to the following pollutants
UVA intensity of 15 Joules/m² for 60 minutes, washed and incubated for 6 hours
UV-B intensity of 4.6 Joule/m² for 30 minutes washed and incubated for 6 hours
Polycyclic aromatic hydrocarbon (Benzo[a]pyrene (BaP) was used at 0.5 mM
Heavy metals (Cobalt chloride and Lead Nitrate at 0.25 mm each)
Intracellular ROS was determined after 6 hours of incubation at 37° C. and 5% CO₂.

### Results

### UV-A

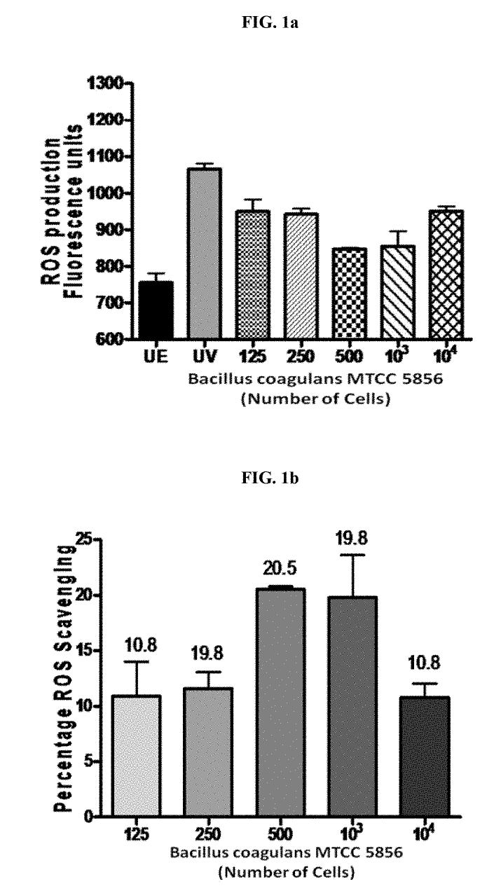

Mouse Fibroblasts were pretreated with Bacillus coagulans MTCC 5856 at different cell numbers for one hour and exposed to UVA at an intensity of 15 Joules/m2 for 30 minutes and reactive oxygen scavenging was recorded. The results indicate that Bacillus coagulans MTCC 5856 conferred protection against UV-A radiation effectively at concentration of 500 cells/well (FIGS. 1a and 1b)

### UV-B

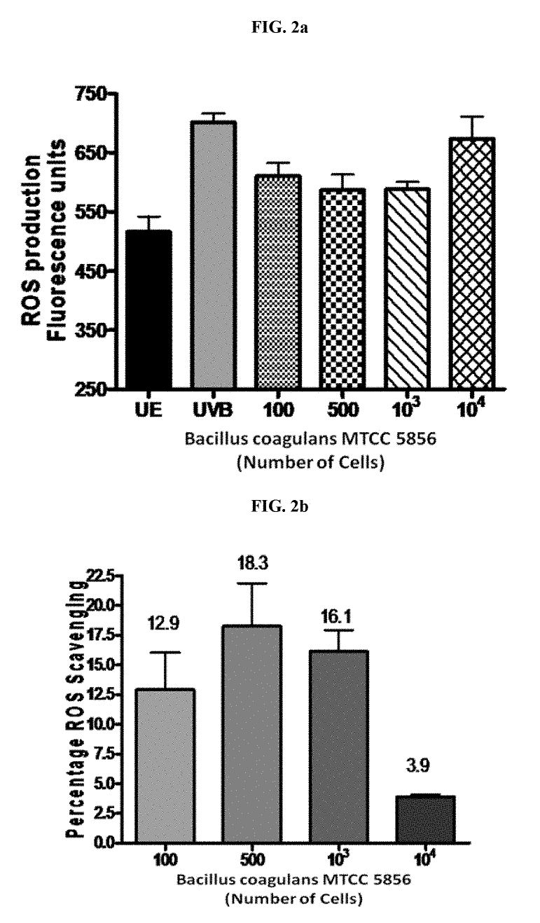

Human Keratinocytes were exposed to UVB irradiation at an intensity of 4.5 Joules/m2 for 10 minutes and reactive oxygen scavenging was recorded. Bacillus coagulans MTCC 5856 at concentrations of 500 cells/well conferred maximum protection by scavenging the ROS produced by 18% (FIGS. 2a and 2b)

### Sodium Lauryl Sulfate

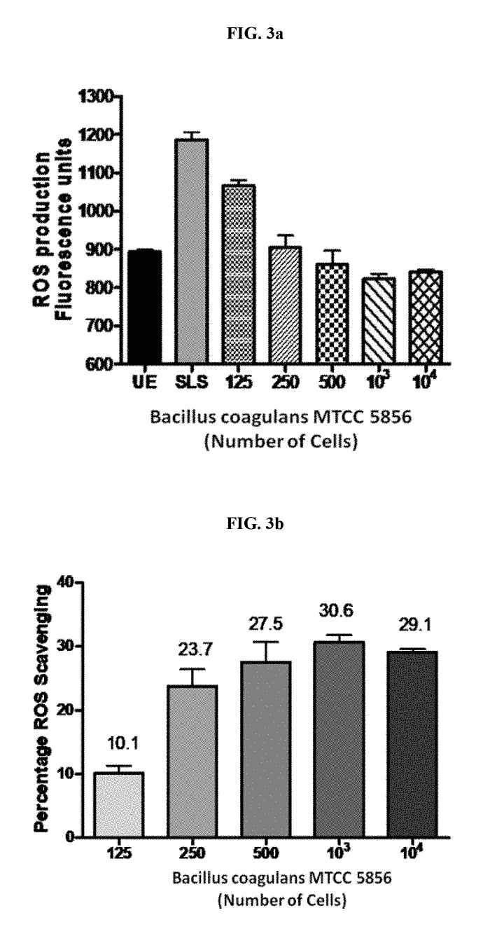

Mouse Fibroblasts were pretreated with Bacillus coagulans MTCC 5856 at different cell numbers for one hour and exposed to sodium lauryl sulfate (SLS) at 300 μM for 60 minutes and reactive oxygen scavenging was recorded. Bacillus coagulans MTCC 5856 exerted antipollution effects by scavenging ROS induced by the detergent in a dose dependant manner (FIG. 3a). Maximum ROS scavenging (30.6%) was observed at cells numbers of 10³cells/well (FIG. 3b)

### Heavy Metals

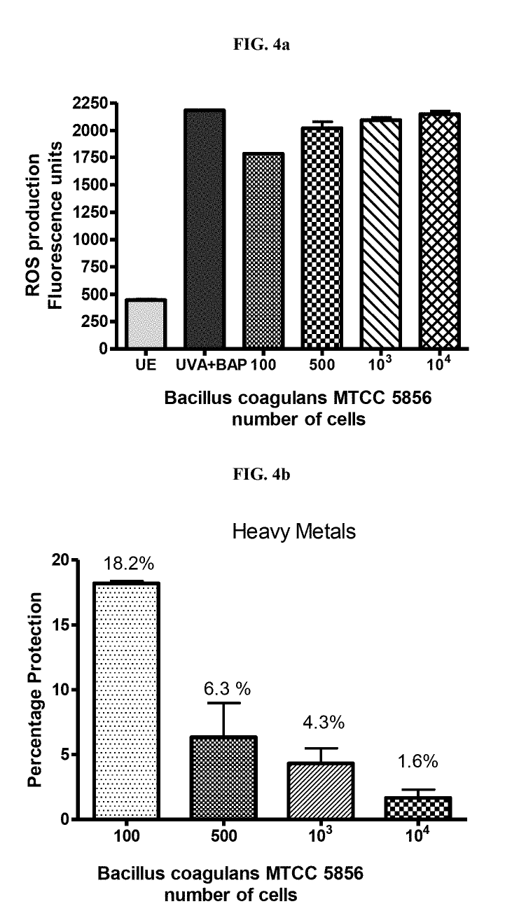

Human Keratinocytes were exposed to Heavy metals (Cobalt chloride and Lead Nitrate at 0.25 mm each) in the presence of different cell numbers of Bacillus coagulans MTCC 5856. Bacillus coagulans MTCC 5856 exerted antipollution effects by scavenging ROS induced by heavy metals (FIG. 4a). Maximum ROS scavenging of 18% was observed at a cell density of 10² cells/well (FIG. 4b).

### Protection Against Polycyclic Aromatic Hydrocarbons in the Presence of UVA

Polycyclic aromatic hydrocarbons (PAHs) are a class of mutagenic and tumorigenic environmental contaminants. PAHs are widespread in the environment produced from incomplete combustion of natural materials and tobacco smoke (Connell, D. W.; Hawker, D. W.; Warne, M. J.; Vowles, P. P.: Polycyclic aromatic hydrocarbons (PAHs). In Introduction into Environmental Chemistry (McCombs, K., and Starkweather, A. W., eds), 1997, pp. 205-217, CRC Press LLC, Boca Raton, Fla. 2. Shaw, G. R.; Connell, D. W.: Prediction and monitoring of the carcinogenicity of polycyclic aromatic compounds (PACs). Rev. Environ. Contam. Toxic., 1994, 135, 1-62.) PAHs themselves are biologically inert and require metabolic activation in order to exert genotoxicity PAHs absorb light in the UVA region. react with oxygen or other molecules to generate reactive intermediates (Yu H, Xia Q, Yan J, et al. Photoirradiation of polycyclic aromatic hydrocarbons with UVA light—a pathway leading to the generation of reactive oxygen species, lipid peroxidation, and dna damage. Int J Environ Res Public Health. 2006; 3: 348-354) Thus, PAHs can be “activated” by light irradiation to cause photo-induced cytotoxicity. Thus photoirradiation of PAHs with UVA irradiation represents a pollutant which causes cytotoxicity and DNA damage.

Human HaCaT keratinocyte cells/mouse fibroblast cells were seeded at a density of 1×10⁴ cells/well in 96 well plates. Cells were allowed to adhere and form a monolayer for 24 hours. They were pretreated with different cell densities of Bacillus coagulans MTCC 5856 for 60 minutes, exposed to UVA at an intensity of 15 Joules/m² in the presence of Benzpyrene a PAH at 0.5 mM for 30 minutes washed with sterile buffer and fresh culture medium (5% of FBS) with respective concentrations of probiotic bacteria Bacillus coagulans MTCC 5856 were added followed by incubation for 6 hours at 37° C. in a CO₂ incubator. Neutral Red (50 pg/mL) (3-amino-7-dimethylamino-2-methylphenazine hydrochloride), was added to the cells for 3 hours. The uptake of NR by the cells was determined by lysing the cells and reading the absorbance at 540 nm in a spectrophotometer (Guidelines, O., Genetic Toxicology: Bacterial Reverse Mutation Assay # 471. 1997)

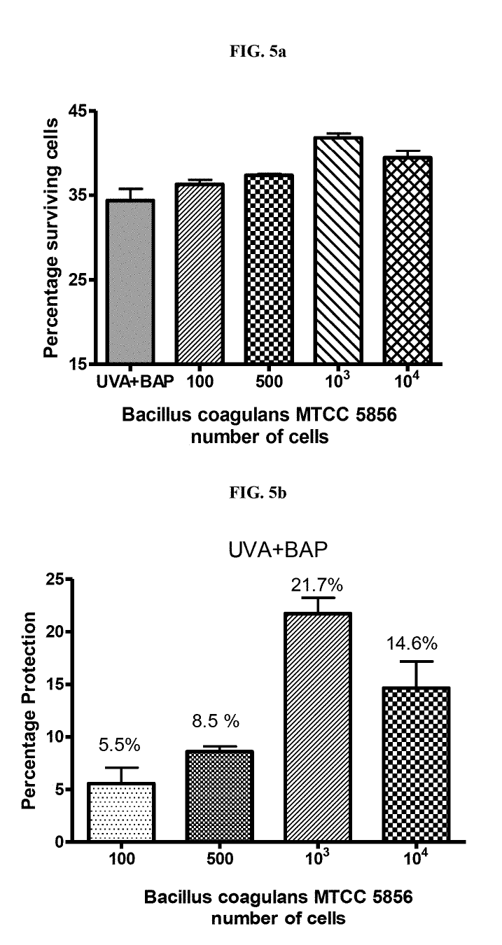

Bacillus coagulans MTCC 5856 exerted antipollution effects by protecting the keratinocytes from cellular cytotoxicity induced by photoirradiation of PAHs with UVA irradiation in a dose dependant manner (FIG. 5a). Maximum ROS scavenging (30.6%) was observed at cells numbers of 10³cells/well (FIG. 5b)

### Example 2

### Effect of Bacillus coagulans MTCC 5856 on Cellular Anti Oxidants

### Methods

Cellular anti oxidants are depleted by pollutants. The ability of Bacillus coagulants MTCC 5856 to increase these anti oxidant enzymes in the cells was studied in vitro. Cellular gultathione (GSH) and superoxide dismutase (SOD) levels were estimated in human keratinocytes (Peskin A V, Winterbourn C C. Assay of superoxide dismutase activity in a plate assay using WST-1. Free Radic Biol Med. 2017; 103:188-191.)

SOD assay The activity of SOD was measured by WST-1 method using a kit as per the manufacturer's instructions (Elabsciences). Xanthine Oxidase (XO) can catalyze WST-1 react with O₂^{.-} to generate a water-soluble formazan dye. SOD can catalyze the disproportionation of superoxide anions, so the reaction can be inhibited by SOD, and the activity of SOD is negatively correlated with the amount of formazan dye. Therefore, the activity of SOD can be determined by the colorimetric analysis of WST-1 products.

Glutathione (GSH) content: Reduced glutathione was determined based on the method of Moron, Depierre. GSH is measured by its reaction with DTNB to give a yellow colored complex with maximum absorption at 412 nm. 100 μl of the test sample (CELL LYSATE) was mixed with 10 μl of 50% TCA was added and centrifuged at 2000 rpm for 10 minute. 30 μl of the supernatant was mixed with 50 μl of 0.2 M sodium phosphate buffer (pH 8.0) and 200 μl of freshly prepared 0.6 mM DTNB and the intensity of yellow colour formation was measured at 412 nm. A standard graph was prepared with different concentrations (1000-62.5 μM) of GSH. The GSH content of the sample was calculated from the standard graph and expressed as μmol/mg protein.

### Results

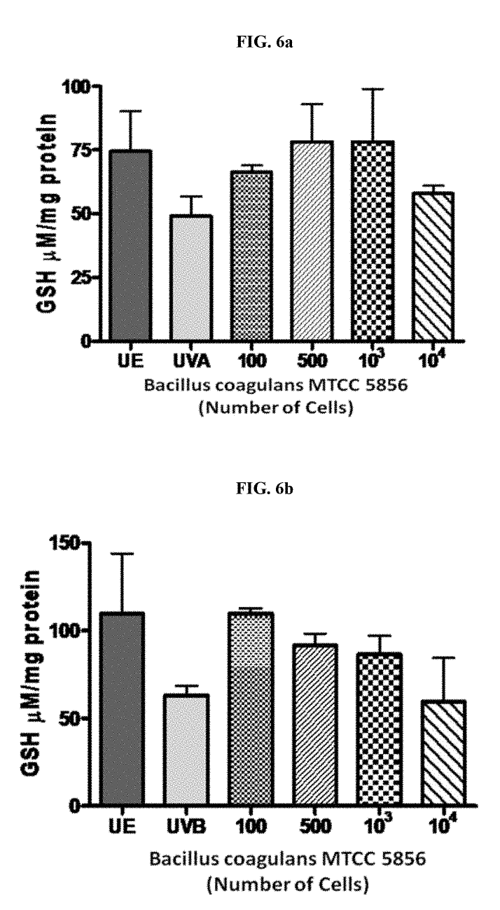

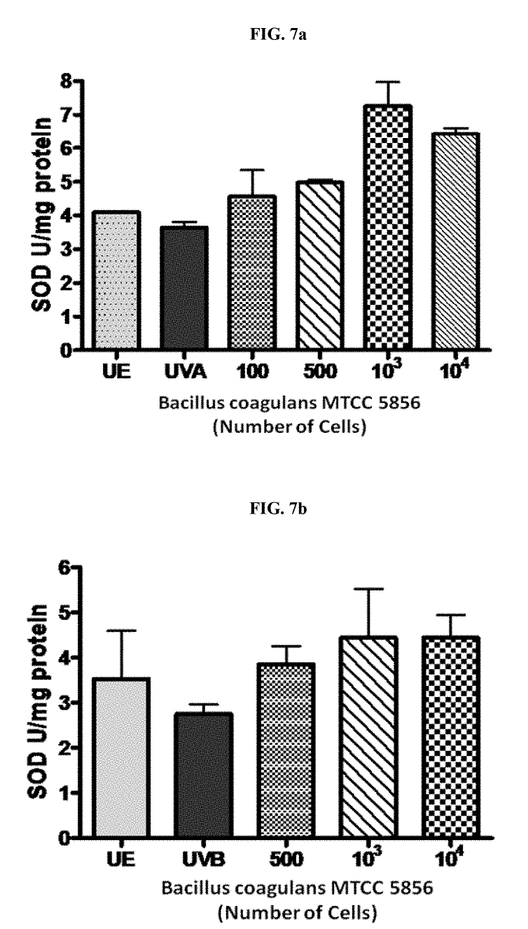

The results revealed that Bacillus coagulans MTCC 5856 increased the glutathione content in a dose dependant manner (FIGS. 6a and 6b). The activity of SOD was also increased in a dose depended manner in cells treated with Bacillus coagulans MTCC 5856 (FIGS. 7a and 7b) indicating that Bacillus coagulans MTCC 5856 is not only an effective antioxidant, but also rejuvenates skin by increasing the antioxidant content in the cells.

### Conclusion

Overall, Bacillus coagulans MTCC 5856 was observed to exert anti-pollution effects by conferring protection against UV and other pollutants by scavenging the ROS produced as a result of exposure to these pollutants. Bacillus coagulans MTCC 5856 also acts as an effective anti-oxidant and also increases the antioxidant content in the cells. The present invention reports that Bacillus coagulans MTCC 5856 can be used not only as an anti-oxidant for the management of different pathological conditions but also as an effective skin rejuvenating and cleansing agent by conferring protection against pollutants and increasing the anti-oxidant content, which can have potential applications in skin care/cosmetic industry.

### Example 3

### Formulations Containing Bacillus coagulans for Skin Care

The composition containing Bacillus coagulans MTCC 5856 may be formulated with pharmaceutically/cosmeceutically acceptable excipients, adjuvants, bases, diluents, carriers, conditioning agents, bioavailability enhancers, antioxidants and preservatives and/or incorporated into formulations containing anti-aging ingredients and administered topically in the form of creams, gels, lotions, powder, serum, oil, suspensions, ointments, soaps, scrubs, emulsions, and compacts.

In a related aspect, one or more skin care ingredients are selected from the group consisting of, but not limited to, Alpha Lipoic Acid, oxyresveratrol, Beet root extract, Boswellia serrata Extract, β boswellic acids, Boswellia serrata oil, Centella asiatica Extract, triterpenes, Garcinia indica extract, anthocyanins, Cocos nucifera extract and juice, Coleus forskohlii Extract, forskolin, Coleus forskohlii Oil, Tetrahydropiperine, Ellagic Acid, Gallnut Extract, polyphenols, Galanga Extract, Glycyrrhizinic Acid, Green Tea Extract, Epigallocatechin Gallate, Licorice extract, MonoAmmonium Glycyrrhizinate, Limonoids, Oleanolic Acid, Cosmetic peptides (Oleanolic acid linked to Lys-Thr-Thr-Lys-Ser, Oleanolic acid linked to Lys-Val-Lys), Oleuropein, Piper longumine extract, piperine, Ellagic acid, Pomegranate Extract (Water Soluble), pterostilbene, resveratrol, Pterocarpus santalinus extract, Rosemary Extract, Rosmarinic Acid, Amla extract, beta glucogallin, tetrahydrocurcumin, Salvia Officinalis (Sage) Leaf Extract, Ursolic Acids, Saponins, Sesamum indicum (Sesame) Seed Extract, Sesamin and sesamolin, moringa oil, moringa seed extract, Horse Chestnut Extract, Vitex Oil, Xymenynic Acid, ethyl ascorbic acid, Argan oil, Lemon peel extract, turmeric oil, Barley Beta Glucans, coenzyme Q10, olive oil, avocado oil and cranberry oil.

In another related aspect, one or more anti-oxidants and anti-inflammatory agents are selected from the group consisting of, but not limited to, vitamin A, D, E, K, C, B complex, rosmarinic acid, Alpha Lipoic Acid, oxyresveratrol, Ellagic Acid, Glycyrrhizinic Acid, Epigallocatechin Gallate, plant polyphenols, Glabridin, moringa oil, oleanolic acid, Oleuropein, Carnosic acid, urocanic acid, phytoene, lipoid acid, lipoamide, ferritin, desferal, billirubin, billiverdin, melanins, ubiquinone, ubiquinol, ascorbyl palmitate, Mg ascorbyl phosphate, ascorbyl acetate, tocopherols and derivatives such as vitamin E acetate, uric acid, α-glucosylrutin, calalase and the superoxide dismutase, glutathione, selenium compounds, butylated hydroxyanisole (BHA), butylated hydroxytoluene (BHT), sodium metabisulfite (SMB), propyl gallate (PG) and amino acid cysteine.

In another related aspect, one or more bioavailability enhancers are selected from the group, but not limited to, piperine, tetrahydropiperine, quercetin, Garlic extract, ginger extract, and naringin.

Tables 1-4 provide illustrative examples of skin care formulations containing Bacillus coagulans MTCC 5856 (commercially available as LACTOSPORE)

TABLE 1
Skin Care Lotion

Active Ingredients
Bacillus coagulans MTCC 5856 100 cfu to 2 billion cfu
Tetrahydrocurcumin, licorice extract, Pterostilbene, Tetrahydropiperine,
Galanga extract, Niacinamide
Other ingredients/Excipients
Aqua, Avobenzone, Octyl methoxy cinnamate, Octocrylene,
Benzophenone-3, Octyl Salicylate, Glyceryl Stearate SE,
Sorbitan Stearate & Sucrose Cocoate, Polysorbate 20,
Glycerin, Cetostearyl Alcohol, Cetearyl Olivate (and) Sorbitan
Olivate, Stearic acid, Isopropyl myristate, Garcinia indica
Seed Butter, Caprylic/Capric Triglyceride, Propylene Glycol,
Butyloctyl Salicylate, Cyclopentasiloxane, Dimethiconol,
Dimethicone Crosspolymer (and) Phenyltrimethicone Blend,
Methylisothiazolinone & Phenoxyethanol, Fragrance,
Cyclotetrasiloxane (and) Cyclopentasiloxane (and) Dimethicone
(and) Trisiloxane (and) Phenyl trimethicone (and) Isoparaffinic
hydrocarbon, Titanium dioxide, Aluminium Hydroxide,
Pentaerithrityl Tetra-di-t-butyl Hydroxyhydrocinnamate,
Pterocarpus marsupium Bark extract}, Tocopheryl
Acetate, Acrylates/C10-30 Alkyl Acrylate Crosspolymer,
Disodium EDTA

TABLE 2
Skin care Hydration Cream

Active Ingredients
Bacillus coagulans MTCC 5856 100 cfu to 2 billion cfu
Amaranthus extract, Niacinamide, Vitamin E, Shea butter, Olive oil, D-
Panthenol
Other ingredients/Excipients
Bioavailability enhancers (Piperine extract or Tetrahydropiperine
(Cosmoperine ®)), Fragrance, Thickeners (Cellulose derivatives or
Acrylates Cross Polymer)

TABLE 3
Cleanser

Active Ingredients
Bacillus coagulans MTCC 5856 100 cfu to 2 billion cfu
Tetrahydrocurcumin, licorice extract, Pterostilbene, Tetrahydropiperine,
Lemon peel extract, papaya extract
Other ingredients/Excipients
Aqua, Sodium cocoyl glycinate, Lauryl Glucoside, Cocamidopropyl
Betaine, Steareth 21, Cetyl alcohol, Carica papaya (Fruit) extract,
Citrus medica limonum (Lemon) Peel Extract, Steareth 2,
PEG-150 Distearate, Propylene Glycol, Acrylates/C10-30 alky acrylate
crosspolymer, Polysorbate 20, Fragrance, Methylisothiazolinone &
Phenoxyethanol, Tocopheryl Acetate, Aminomethyl Propanol, Mica
(and) CI 77891, Pentaerithrityl Tetra-di-t-butyl Hydroxyhydrocinnamate,
Disodium EDTA, bioavailability enhancers (Piperine extract or
Tetrahydropiperine (Cosmoperine ®)), Fragrance, Thickeners
(Cellulose derivatives or Acrylates Cross Polymer)

TABLE 4
Face Scrub

Active Ingredients
Bacillus coagulans MTCC 5856 100 cfu to 2 billion cfu
Cocus nucifera extract, walnut scrub, neem oil, Niacinamide,
lemon peel extract, Vitamin E acetate
Other ingredients/Excipients
Aqua, Glycerin, Caprylic/Capric Triglyceride, Lauryl Glucoside, Sorbitan
Stearate & Sucrose Cococate, Isopropyl Myristate, Isopropyl Palmitate &
Pentaerthrityl Tetraisostearate, Cetyl Palmitate, Stearic acid, Cetostearyl
Alcohol, CI 77891, Juglans Regia (Walnut) Shell Powder, Zea Mays
(corn) Starch, Azadirachta Indica (Neem) Seed Oil, Phenoxyethanol &
Methylisothiazolinone, Fragrance, Acrylates/C10-30 Alkyl Acrylate
Crosspolymer, Amino Methyl Propanol, Pentaerthirtyl Tetra-di-t-butyl
Hydroxyhydrocinnmate, Tocopheryl Acetate, Menthol, Disodium EDTA,
bioavailability enhancers (Piperine extract or Tetrahydropiperine
(Cosmoperine ®)), Fragrance, Thickeners (Cellulose
derivatives or Acrylates Cross Polymer)

### Example 5

### Formulations Containing Bacillus coagulans for General Health

Tables 5 and 6 provide illustrative examples of formulations containing Bacillus coagulans for use an antioxidant and maintaining the redox equilibrium of the cells.

TABLE 5
Bacillus coagulans Tablet

Active Ingredients
Bacillus coagulans MTCC 5856: 2 billion cfu
Excipients
Microcrystalline cellulose, Colloidal silicon dioxide, Magnesium stearate

TABLE 6
Bacillus coagulans Capsule

Active Ingredients
Bacillus coagulans MTCC 5856: 2 billion cfu
Excipients
Microcrystalline cellulose

The above formulations are merely illustrative examples; any formulation containing the above active ingredient intended for the said purpose will be considered equivalent.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention. The scope of the invention is to be interpreted only in conjunction with the appended claims.

We claim:

1. A composition containing probiotic bacteria Bacillus coagulans for protecting mammalian skin against the harmful effects of UV radiation and environmental pollutants.

2. The composition as in claim 1, wherein the probiotic bacteria Bacillus coagulans is present in the form of a spore or viable bacilli.

3. The composition as in claim 1, wherein the environmental pollutants are selected from the list consisting of, but not limited to, particulate matter, polycyclic aromatic hydrocarbons (PAHs), volatile organic compounds (VOCs), detergents, nitrogen and sulfur oxides, carbon monoxide, ozone, and heavy metals.

4. The composition as in claim 1, wherein the probiotic bacteria confers skin protection by increasing the levels of anti-oxidants and decreasing ROS levels.

5. The composition as in claim 1, wherein the Bacillus coagulans strain is preferably Bacillus coagulans MTCC 5856.

6. The composition as in claim 1, wherein the composition is formulated with pharmaceutically/cosmeceutically acceptable excipients, adjuvants, bases, diluents, carriers, conditioning agents, bioavailability enhancers, and preservatives and/or incorporated into formulations containing skin care ingredients and administered topically in the form of creams, gels, lotions, powder, serum, oil, suspensions, ointments, soaps, scrubs, emulsions, and compacts.

7. A method for cleansing and rejuvenating mammalian skin, exposed to environmental pollutants and UV radiation, said method comprising step of administering an effective dose of a composition containing probiotic bacterial Bacillus coagulans to mammals in need of such effect.

8. The method as in claim 7, wherein the probiotic bacteria Bacillus coagulans is present in the form of a spore or viable bacilli.

9. The method as in claim 7, wherein the environmental pollutants are selected from the list consisting of, but not limited to, particulate matter, polycyclic aromatic hydrocarbons (PAHs), volatile organic compounds (VOCs), detergents, nitrogen and sulfur oxides, carbon monoxide, ozone, and heavy metals.

10. The method as in claim 7, wherein the probiotic bacteria rejuvenates the skin by increasing the levels of anti-oxidants and decreasing ROS levels.

11. The method as in claim 7, wherein the Bacillus coagulans strain is preferably Bacillus coagulans MTCC 5856.

12. The method as in claim 7, wherein the composition is formulated with pharmaceutically/cosmeceutically acceptable excipients, adjuvants, bases, diluents, carriers, conditioning agents, bioavailability enhancers, and preservatives and/or incorporated into formulations containing skin care ingredients and administered topically in the form of creams, gels, lotions, powder, serum, oil, suspensions, ointments, soaps, scrubs, emulsions, and compacts.

13. A composition containing probiotic bacteria Bacillus coagulans for use as an antioxidant.

14. The composition as in claim 13, wherein the composition containing probiotic bacteria Bacillus coagulans is used in the therapeutic management of mammalian cellular oxidative stress.

15. The composition as in claim 13, wherein the probiotic bacteria Bacillus coagulans is present in the form of a spore or viable bacilli.

16. The composition as in claim 13, wherein the Bacillus coagulans strain is preferably Bacillus coagulans MTCC 5856.

17. The composition as in claim 13, wherein composition is formulated with pharmaceutically/cosmeceutically acceptable excipients, adjuvants, bases, diluents, carriers, conditioning agents, bioavailability enhancers, and preservatives and/or incorporated into formulations containing skin care ingredients and administered orally or topically in the form of tablet, capsule, powder, emulsions, solution, creams, gels, lotions, powder, serum, oil, suspensions, ointments, soaps, scrubs, emulsions, and compacts, suited for nutraceutical, cosmeceutical and nutri-cosmetic applications.
